(19) United States
(12) Patent Application Publication
(10) Pub. No.: US 2013/0122468 A1
(43) Pub. Date: May 16, 2013

(54) METHOD OF PROCESSING AND DISPLAYING ORAL HEALTH DIAGNOSTIC DATA

(71) Applicants: Stephen Abrams, Toronto (CA); Koneswaran Sivagurunathan, Scarborough (CA); Josh Silvertown, Toronto (CA); Jin-Seok Jeon, Windsor (CA); Andreas Mandelis, Scarborough (CA)

(72) Inventors: Stephen Abrams, Toronto (CA); Koneswaran Sivagurunathan, Scarborough (CA); Josh Silvertown, Toronto (CA); Jin-Seok Jeon, Windsor (CA); Andreas Mandelis, Scarborough (CA)

(73) Assignee: QUANTUM DENTAL TECHNOLOGIES, INC., Toronto, ON (CA)

(21) Appl. No.: 13/697,758

(22) PCT Filed: May 13, 2011
(86) PCT No.: PCT/CA2011/050302

§ 371 Date: Jan. 25, 2013

Related U.S. Application Data
(60) Provisional application No. 61/334,415, filed on May 13, 2010.

(51) Int. Cl. A61B 5/00

(52) U.S. Cl. CPC A61B 5/4547
USPC 433/215

(57) ABSTRACT

A method is provided for analyzing and displaying an oral health status of a tooth or an entire dentition based on a measurement with a diagnostic device. Diagnostic data pertaining to a selected tooth, tooth surface, section of tooth surface or numbers of teeth in a mouth is recorded from an oral health diagnostic device, optionally along with an image of the particular tooth or tooth surface examined. The diagnostic data is processed and compared with reference data to determine an oral health status of the tooth. The oral health status of the tooth is then displayed on an odontogram shown in a user interface. The user interface may also provide reports comparing changes in the measured data and/or images along with the therapies used, thereby enabling the measurement and tracking of outcomes from various therapies over time.

### CROSS-REFERENCE TO RELATED APPLICATION

This application claims priority to U.S. Provisional Application No. 61/334,415 titled “Method of Processing and Displaying Oral Health Diagnostic Data” and filed on May 13, 2010, the entire contents of which are incorporated herein by reference.

### BACKGROUND

The present disclosure relates to diagnostic methods in dentistry and oral health care, and more particularly, the present disclosure relates to methods of processing and presenting results from oral diagnostic procedures.

With the widespread use of fluoride, the prevalence of dental caries has been considerably reduced. Nonetheless, the development of a non-invasive, non-contact technique that can detect and monitor early demineralization and or carious lesions on or beneath the enamel, dentin, root surface or dental restorations, is essential for the clinical management of this problem. A number of different diagnostic devices and methods have been developed to meet this need, including laser-induced fluorescence of enamel or to the fluorescence caused by porphyrins present in carious tissue [R. Hibst, K. Konig, “Device for Detecting Dental Caries”, U.S. Pat. No. 5,306,144 (1994)] and photothermal radiometry [A. Mandelis, L. Nicolaides, C. Feng, and S. H. Abrams, “Novel Dental Depth Profilometric Imaging Using Simultaneous Frequency-Domain Infrared Photothermal Radiometry and Laser Luminescence”, Biomedical Optoacoustics. Proc SPIE, A. Oraevsky (ed), 3916, 130-137 (2000), L. Nicolaides, A. Mandelis, and S. H. Abrams, “Novel Dental Dynamic Depth Profilometric Imaging Using Simultaneous Frequency-Domain Infrared Photothermal Radiometry and Laser Luminescence”, J Biomed Opt, 5, 31-39 (2000), and R. J. Jeon C. Han A. Mandelis V. Sanchez S. H. Abrams “Diagnosis of Pit and Fissure Caries using Frequency Domain Infrared Photothermal Radiometry and Modulated Laser Luminescence” Caries Research 38, 497-513 (2004)] smooth surface and interproximal lesion detection].

While these oral health diagnostic devices succeed in providing quantitative measures of existing and anticipated oral health decay, their results are often not directly amenable to clinical practice. Firstly, the recording of numerical data based from a diagnostic device presents a workflow challenge to an oral health provider, and the manual recording of results is susceptible to transcription errors that could result in costly or inappropriate treatment. Secondly, describing and transcribing the status of oral tissues including exact colour, shape and position of a pathological condition is most challenging and may lead to inaccuracies and inability to track changes in the tissues over time. Furthermore, merely sharing a numerical value provided by a diagnostic device with a patient offers little insight to the patient in terms of the severity of a problem. Such raw and direct results do not assist in providing a path that the patient and provider can take together to manage a given condition and/or mitigate risks of developing an oral health problem in the future.

### SUMMARY

Embodiments provided herein disclose a method for calculating, monitoring, tracking and displaying an oral health status of a tooth, section of a tooth or an entire dentition, based on a measurement with a diagnostic device. Diagnostic data pertaining to a selected tooth, tooth surface, section of tooth surface, or numbers of teeth in a mouth, is recorded from an oral health diagnostic device, optionally along with an image of the particular tooth or tooth surface examined. The diagnostic data is processed and compared with reference data to determine an oral health status of the tooth. The oral health status of the tooth is then displayed on an odontogram or other report shown in a user interface. The user interface may also provide reports comparing changes in the measured data and/or images along with the therapies used, thereby enabling the measurement and tracking of outcomes from various therapies over time.

Accordingly, in one aspect, there is provided a computer implemented method of displaying oral health status information of a patient, the method comprising the steps of receiving, from an oral health diagnostic device, diagnostic data pertaining to two or more surfaces of a selected tooth; processing the diagnostic data for each surface of the one or more surfaces to determine an oral health status of the selected tooth; and displaying, a user interface, an odontogram comprising an indication of the oral health status of the selected tooth.

In another aspect, there is provided a system for displaying an oral health status of a selected tooth or tooth surface, the system comprising: an interface for receiving diagnostic data pertaining to the selected tooth or tooth surface from an oral health diagnostic device; a processor programmed to: compare the diagnostic data to reference data and infer an oral health status of the selected tooth or tooth surface; display a user interface comprising an odontogram; and displaying an indication of the oral health status of the selected tooth (based upon the analysis of all data from one or more surfaces of the tooth) on the odontogram; and a display for displaying the user interface. The system may include the oral health diagnostic device.

A further understanding of the functional and advantageous aspects of the disclosure can be realized by reference to the following detailed description and drawings.

### BRIEF DESCRIPTION OF THE DRAWINGS

Embodiments will now be described, by way of example only, with reference to the drawings, in which:

FIG. 1 provides a flowchart illustrating a method of displaying an odontogram comprising results from a diagnostic oral health device;

FIG. 2 illustrates an odontogram with integrated results from a photothermal radiometric and luminescence (PTR-LUM) device on several scanned teeth;

FIG. 3 is a screenshot from a user interface displaying an odontogram adjacent to a treatment note;

FIG. 4 is a block diagram illustrating various screens accessible via the user interface;

FIG. 5(a) illustrates a touchscreen user interface for displaying the oral health status of selected teeth;

FIG. 5(b) illustrates a touchscreen user interface displaying the results of the scanning examination.

FIG. 6 provides screenshots showing (a) the scanning of a selected tooth, with the selected tooth identified on the odontogram adjacent to an image of the tooth, (b) the scanning of a particular region on the surface of a tooth pertaining to an element of a grid and (c) shows a report from examining a tooth surface;

FIG. 7 is a photograph illustrating locations for performing interproximal areas;

FIG. 8 provides a flowchart illustrating a method of acquiring an image of a tooth and subsequently performing a scan of the imaged tooth;

FIG. 9 is an example screenshot illustrating the selection and generation of a report;

FIG. 10 provides an example of a report generated by the system;

FIG. 11 is a schematic of a local computing system for receiving, processing and displaying diagnostic data on an odontogram;

FIG. 12 is a schematic of a network comprising a computing system for receiving, processing and displaying diagnostic data on an odontogram on a remote workstation;

FIG. 13 is a schematic block diagram of an embodiment of the dental diagnostic device forming part of the dental data management system;

FIG. 14 provides (a) a schematic block diagram of an embodiment of the hand piece forming part of the dental diagnostic device and dental data management system, and (b) a schematic showing the internal components of a handpiece with an integrated camera; and

FIG. 15 shows (a) a graph plotting the time dependence of the Canary Number measured over four separate visits, and (b) images of the tooth indicating the surface area scanned.

### DETAILED DESCRIPTION

Various embodiments and aspects of the disclosure will be described with reference to details discussed below. The following description and drawings are illustrative of the disclosure and are not to be construed as limiting the disclosure. Numerous specific details are described to provide a thorough understanding of various embodiments of the present disclosure. However, in certain instances, well-known or conventional details are not described in order to provide a concise discussion of embodiments of the present disclosure.

As used herein, the terms, “comprises” and “comprising” are to be construed as being inclusive and open ended, and not exclusive. Specifically, when used in the specification and claims, the terms, “comprises” and “comprising” and variations thereof mean the specified features, steps or components are included. These terms are not to be interpreted to exclude the presence of other features, steps or components.

As used herein, the term “exemplary” means “serving as an example, instance, or illustration,” and should not be construed as preferred or advantageous over other configurations disclosed herein.

As used herein, the term “diagnostic data” means data that relates to a measurement that is performed by an oral health detection or evaluation device or an oral health tool.

As used herein, the terms “about” and “approximately”, when used in conjunction with ranges of dimensions of particles, compositions of mixtures or other physical properties or characteristics, are meant to cover slight variations that may exist in the upper and lower limits of the ranges of dimensions so as to not exclude embodiments where on average most of the dimensions are satisfied but where statistically dimensions may exist outside this region. It is not the intention to exclude embodiments such as these from the present disclosure.

In one embodiment, a method is provided for the processing and display of data relating to a measurement made by an oral health diagnostic/detection device.

The oral health diagnostic device is used for capturing of data indicative of the health or disease present in a tooth, section of tooth and supporting structure (hard and soft tissues in the oral cavity) including information on dental caries, cracks, erosion lesions, restorations, integrity of restorations, periodontal disease and other diseases of the hard and soft tissues. In one embodiment, the data is obtained by scanning a tooth surface or section of a tooth surface using the dental diagnostic device for the detection and monitoring of dental caries, erosion, secondary caries or caries around the margins of restorations and capturing of this data and other relevant information used in the dental diagnostic device. This data is then stored in a device in association with identifying information such as patient ID, demographic data, tooth and/or site examined. Several non-limiting examples of oral health detection devices are provided below.

The oral health diagnostic data may include quantitative data correlated with the presence or absence of one or more oral health conditions. Example non-limiting conditions include of demineralization of teeth, remineralization of teeth, presence of dental caries on enamel surfaces, presence of dental caries on root surfaces, erosion, defects in restorations, defects and caries along the margins of restorations, cracks, periodontal disease, diseases of the hard and soft tissues, and oral cancer. Additionally, the device may detect changes associated with the health of a tooth, such as demineralization or caries on the enamel surface, demineralization or caries on the root surface, remineralization of the root surface, remineralization of the enamel surface, and restoration in or on the tooth or its surrounding tissue. Those skilled in the art will appreciate that a wide variety of oral health detection devices are compatible with embodiments of the disclosure.

The oral health diagnostic device may employ an optical signal for the measurement of a dental health condition. Such optical signals include, but are not limited to, luminescence, fluorescence, and/or thermal emission. Such optical signals may be at various frequencies. Many biological objects containing fluorescing components (fluorophores) exhibit intrinsic fluorescence (or autofluorescence). In dentistry, the aim of recent scientific research has been the use of laser fluorescence for detection of tooth demineralization or caries on enamel and/or root surfaces, dental deposits, and dental calculus and quantitative analysis of lesion depth and size, as well as the mineral composition of the enamel [M. L. Sinyaeva, Ad. A. Mamedov, S. Yu. Vasilchenko, A. I. Volkova, and V. B. Loschenov, 2003, “Fluorescence Diagnostics in Dentistry”, Laser Physics, 14, No. 8, 2004, pp. 1132-1140].

UV radiation (488 nm) has been used to examine dental enamel [Susan M. Higham, Neil Pender, Elbert de Josselin de Jong, and Philip W. Smith, 2009. Journal of Applied Physics 105, 102048, R. Hibst and R. Paulus, Proc. SPIE 3593, 141 (1999)]. The studies showed that autofluorescence of healthy enamel were peaked at a wavelength of 533 nm, whereas the autofluorescence of carious tissue was red-shifted by 40 nm. It was also demonstrated that the autofluorescence intensity of carious zones was an order-of-magnitude lower than the autofluorescence intensity of a healthy tooth in spite of the fact that the absorbance of the carious zone at the excitation wavelength was significantly higher.

The reduction in fluorescence when enamel demineralizes or develops a carious lesion has been attributed to the increase in porosity of carious lesions when compared with sound enamel. There is an associated uptake of water and decrease in the refractive index of the lesion resulting in increased scattering and a decrease in light-path length, absorption, and autofluorescence [H. Bjelkhagan, F. Sundström, B. Angmar-M\check{a}nsson, and H. Ryder, Swed Dent. J. 6, 1982].

At long wavelengths excitation, the autofluorescence intensity of a carious cavity can be higher than the autofluorescence intensity of healthy tissue [R. Hibst et al.]. For excitation wavelengths of 640 or 655 nm, the integral (at wavelengths greater than 680 nm) autofluorescence intensity of a carious cavity could be approximately one order-of-magnitude greater than the corresponding integral autofluorescence intensity of healthy enamel. There is some indication that the induced fluorescence with these wavelengths results from the excitation of fluorescent fluorophores from bacterial metabolites. These fluorophores are thought to originate from porphyrins found in some bacterial species [S. M. Higham et al.] but not the primary bacterial species (Strep Mutans and Lactobaccli) that are the causative organisms in dental caries.

Accordingly, in one embodiment, the diagnostic data may be provided by an oral health detection device such as, but not limited to, commercial dental diagnostic systems such as those offered by QLF™ and DIAGNOdent™.

More recently, a new system has been developed based on the combination of laser induced fluorescence and photothermal radiometry. The system, commercially available as The Canary System™, which examines luminescence and photothermal effect (PTR-LUM) of laser light on a tooth, as described in US Patent Application No. 2007/0021670, titled “Method and Apparatus Using Infrared Photothermal Radiometry (PTR) and Modulated Laser Luminescence (LUM) for Diagnostics of Defects in Teeth”, filed Jul. 18, 2006, which is herein incorporated by reference in its entirety. The laser is non-invasive and can detect tooth decay a fraction of a millimeter in depth and up to five millimeters below a tooth's surface.

When pulses of laser light are focused on a tooth, the tooth glows and releases heat. By analyzing the emitted light and heat signatures from the tooth, very accurate information about the tooth's condition can be obtained including signs of early demineralization (carious lesions) of enamel or root surface [Nicolaides, L, Mandelis, A Abrams, S. H “Novel Dental Dynamic Depth Profilometric Imaging using Simultaneous Frequency Domain Infrared Photothermal Radiometry and Laser Luminescence”, Journal of Biomedical Optics, 2000, January, Volume 5, #1, pages 31-39, Jeon, R. J., Han, C., Mandelis, A., Sanchez, V Abrams, S. H., “Non-intrusive, Non-contacting Frequency-Domain Photothermal Radiometry and Luminescence Depth Profilometry of Carious and Artificial Sub-surface Lesions in Human Teeth,” Journal of Biomedical Optics 2004, July-August, 9, #4, 809-81, Jeon R. J., Hellen A Matvienko A. Mandelis A Abrams S. H. Amaechi B. T., In vitro Detection and Quantification of Enamel and Root Caries Using Infrared Photothermal Radiometry and Modulated Luminescence. Journal of Biomedical Optics 13(3), 048803, 2008]. As a lesion grows, there is a corresponding change in the signal. As remineralization progresses, a signal reversal indicates an improvement in the condition of the tooth. By changing the frequency of the signal one can probe up to 5 mm below the tooth surface. Low frequency signals can penetrate the defects and lesions beneath the tooth surface.

One example implementation of a diagnostic device is a hybrid PTR-LUM system, which may be a phase-sensitive detection system that performs four measurements per location and/or per frequency at each location:
1. PTR Amplitude: the strength of the emitted blackbody IR signal
2. LUM Amplitude: the strength of the luminescence signal
3. PTR Phase: the shift in phase of the emitted blackbody IR signal
4. LUM Phase: the shift in phase of the luminescence signal
   These four measurements, when combined, provide information on the status of the tooth surface and changes in the carious lesion. Example 1 below provides an example implementation for combining the PTR and LUM data. Alternatively, a subset of the above measurements may be combined for use with the above method, for example, combining PTR amplitude data and LUM amplitude data.

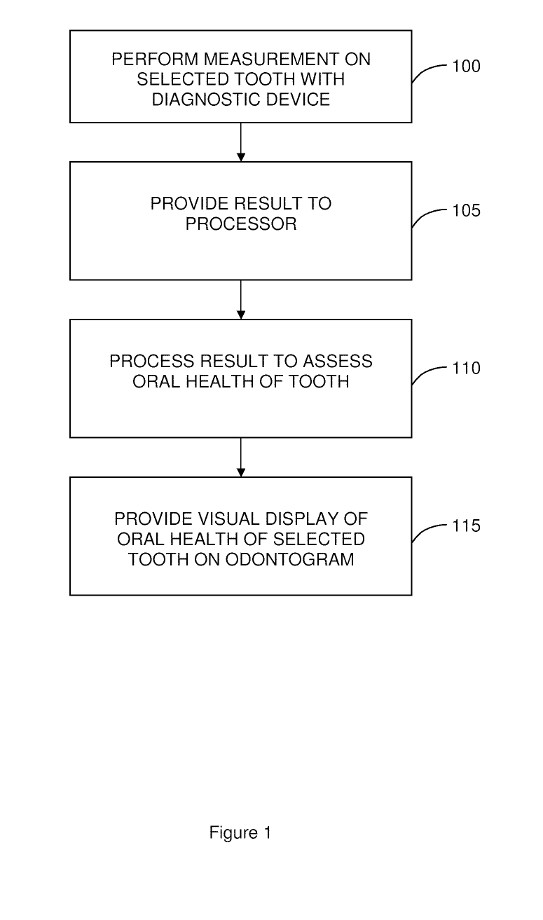

Referring now to FIG. 1, a measurement is performed on a selected tooth at step 100 using an oral health diagnostic device. The device may be any diagnostic device that provides a measure related to the oral health status of a specific tooth, such as the example provided above. In step 105, the measured data is provided to a processor, such as a computer or data acquisition card. The data may comprise any type of measured data, including raw data and pre-processed data. For example, the data may be normalized or otherwise processed by an additional processor or computing system located within the diagnostic device, or within a subsystem of the diagnostic device, prior to being provided to the processor in step 105.

The data is subsequently processed by the processor in step 110 to provide an indication of the oral health status of the tooth based on the measured data. This step involves the comparison of the measured data, for each measured surface, with a reference value to determine whether the measured data corresponds to a healthy tooth or if the measured data is characteristic of an oral health condition or a risk of developing an oral health condition. The reference data may comprise a multitude of forms, including, but not limited to, reference data provided by the device manufacturer indicative of healthy and unhealthy oral health status of a tooth, reference data based on measurement of reference samples, reference data based on analysis of a patient population, and reference data obtained based on published studies.

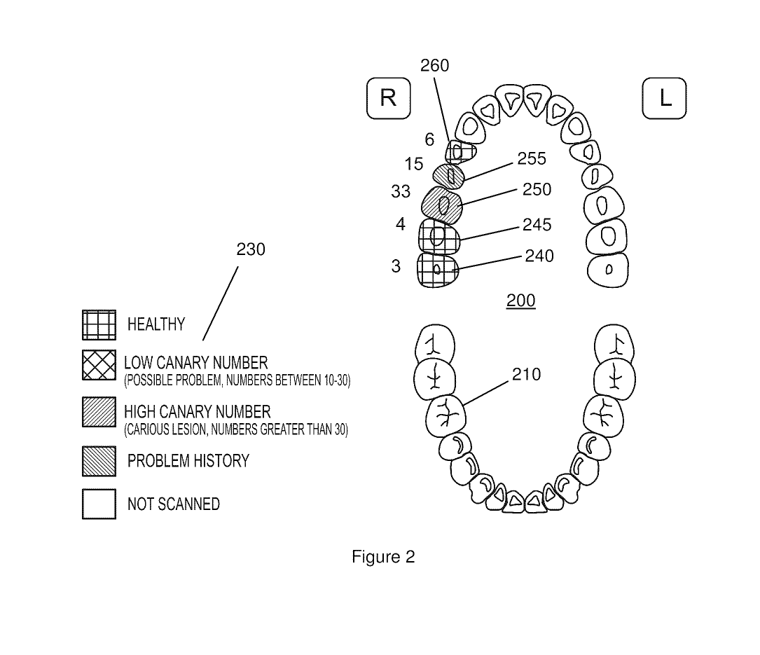

In step 115, an odontogram is displayed in a graphical user interface for the provider and/or patient to observe, assess or review the oral health status of the patient's teeth, and a visual indication of the oral health status of the measured tooth is provided. FIG. 2 illustrates an example odontogram 200 that includes a chart showing the upper and lower jaw with teeth 210 of a patient. The odontogram may be a basic odontogram as shown in FIG. 2, or may comprise additional oral health information such as existing problems, restorations including fillings implants and or crowns, pocket depths and recession readings. The odontogram may display the permanent dentition, primary dentition, supernumerary dentition or mixed dentition. Clicking or touching a tooth may activate the primary tooth replacement (if in the permanent dentition) or the permanent tooth replacement if in the primary dentition.

It is to be understood that the odontogram shown in FIG. 2 is merely one example of an odontogram, and that many other representations a patient's teeth are possible and intended to lie within the scope of the present disclosure. Odontograms may take various forms and levels of abstraction for showing a visual representation of a patient's teeth. Another example odontogram provides a representation in the form of parallel rows of teeth, instead of the oval shaped representation shown in FIG. 2.

In one embodiment, two or more surfaces of a selected tooth are measured (e.g. scanned), and the multiple readings may be combined when determining the oral health status for the tooth. For example, the highest value of the scanned values obtained for the given tooth may be employed when determining the oral health status (for example, the highest value obtained may be compared with a reference value). In another example, the values may be averaged when determining the oral health status. In yet another example, the multiple readings may be processed such that an integrated diagnostic value is provided that is related to the number of readings above a certain threshold. In another example, multiple ranges, defined by two or more thresholds, may be employed in the calculation of the integrated diagnostic value, and where the integrated diagnostic value is determined by calculating the weighted sum of the number of measurements within each range (the resulting sum may be normalized to a suitable value).

In the example embodiment shown in FIG. 2, the oral health status determination is made by binning the diagnostic measurement into one of several categories indicative of various degrees of oral health. FIG. 2 shows a particular and non-limiting example in which the various categories shown in legend 230 are classified by the Canary number, which is a unified measure of PTR-LUM data measurements as described above. The oral health is classified by the magnitude of the Canary number, where Canary values are binned as follows:

0-10: Healthy

10-30: Possible Oral Health Problem

>30: Carious Lesion

As shown in legend 230, known problems based on the patient's oral health history may also be optionally shown in odontogram 200.

Odontogram 200 shows results of four scanned teeth, with various results based on the magnitude of the Canary reading. As shown, the Canary readings, or more generally, the measured values from the diagnostic device, may be shown on the odontogram to further assist in the interpretation of the results. Teeth 240 and 245 have low associated Canary numbers of 3 and 4, respectively, suggestive of good oral health. Tooth 250 has a high Canary number of 33, which is above the threshold for indicating poor oral health, such as a carious lesion. Tooth 255, which has a Canary number of 15, is suggestive of a possible oral health problem, or a risk of developing a future oral health condition such as a carious lesion. However, according to odontogram 200, tooth 255 also has an associated known problem history, therefore meriting clinical concern and attention. Tooth 260, like teeth 240 and 245, appears to be healthy based on its low Canary reading.

While the example shown in FIG. 2 illustrates the visual display of various oral health conditions of measured teeth based on colouring or patterning the teeth as displayed, it should be understood that a wide variety of visual indicators are contemplated by embodiments of the present disclosure. In several non-limiting examples, the oral health status of a measured tooth may be indicated by textual markings adjacent to the tooth, the fill or outline colour of a tooth, the fill pattern of a tooth, the intensity of the displayed tooth outline, and the intensity of the fill of a tooth.

While the example embodiment shown in FIG. 2 provides a binned categorization of the oral health status, other embodiments may be provided in which the oral health status is displayed using a continuous range. Such a continuous range may be displayed, in non-limiting examples, by shades of a colour or different continuous levels of intensity of a displayed tooth, tooth outline, or adjacent textual label.

In one embodiment, the oral health status is determined by a combination of the measured diagnostic data, and patient risk factor data, as taught in co-pending U.S. patent application Ser. No. 12/718,746, titled “Method of Assessing Oral Health Risk” and filed on Mar. 5, 2010, which is herein incorporated by reference in its entirety. The patient risk factor data may be provided as additional input that is separate from the diagnostic device data (although the data can be input by an input device connected to the diagnostic device, such as a keyboard and mouse, voice activated software and data input systems). The risk factor data thus provides information regarding additional tertiary factors (such as subjective risk factors) that can impact the risk of developing an oral health condition.

In one example implementation, the patient risk factors may be categorized into two or more groups, such as pathological risk factors, protective risk factors, historical factors, behavioural and/or extrinsic factors. The pathological risk factors may include, but are not limited to, a plaque index, quantity of existing tooth decay, size of existing tooth decay, distribution of existing tooth decay, presence of acidogenic or pathologic bacteria, reduced salivary flow, bleeding of gums when brushed or flossed, number of decayed, missing or filled teeth, numbers of decayed missing and filled tooth surfaces, crowding or mal-alignment of the teeth and frequency of carbohydrate ingestion. The historical risk factors may include, but are not limited to, an integrity of a tooth surface, a status of oral tissues, a history of grinding or clenching or bruxing of the teeth, exposed root surfaces, number of years living in a fluoridated community, and a number within a prescribed period of fillings, root canals, crowns, bridges, partial dentures, tooth extractions, oral and periodontal surgical procedures and implants.

The protective risk factors may include, but are not limited to, use of remineralization agents, an amount of salivary flow, the presence of salivary components comprising one or more of proteins, calcium, phosphate, fluoride, immunoglobins, and antibacterials in saliva. Behavioral risk factors may include, but are not limited to, chewing gums and consumption of dairy products, consumption of carbohydrates and tendency to grind teeth.

Self-care risk factors may include, but are not limited to, frequency of tooth brushing, timing of oral health maintenance including brushing or flossing, frequency of tooth flossing, manual dexterity and ability to properly use various oral health aids properly including a tooth brush, use of a fluoridated toothpaste, use of other oral health home care aids, and use of selected mouth rinses.

Furthermore, the extrinsic risk factors may include, but are not limited to, diet, sufficiency of home care, access to oral care, gender, age, geographic location, socio-economic status and one or more demographic factors.

As described in co-pending U.S. patent application Ser. No. 12/718,746, patient risk measures related to the patient risk factor data may be determined by the processor carrying out a series of computational steps. Patient risk factor data may be compared to pre-determined risk-associated risk factor values to obtain the patient risk measures. In one example implementation, the patient risk measures may be obtained by comparing the risk factor data to pre-determined risk factor values and obtaining a risk score based on the comparison.

The patient risk measures and the diagnostic data obtained from the diagnostic device for a specific tooth or portion of a tooth surface may then be processed to obtain a single integrated risk measure for the specific tooth. In one non-limiting example implementation, a numerical value is attributed to each patient data risk measure, and the values for each risk measure and the diagnostic results are weighted and averaged to obtain the integrated risk measure. The risk measures may be weighted prior to being processed in order to obtain a clinically significant integrated risk measure.

Similar to the aforementioned case where more than one location of a tooth surface is scanned and where multiple diagnostic measurements are obtained, the multiple readings may be combined when determining the integrated risk measure for the tooth. For example, the highest value of the scanned values obtained for the given tooth may be employed when determining the integrated risk measure. In another example, the values may be averaged when determining the integrated risk measure. In yet another example, the multiple readings may be processed such that an integrated diagnostic value is provided that is related to the number of readings above a certain threshold, and the integrated diagnostic value is employed when determining the integrated risk measure. In another example, multiple ranges, defined by two or more thresholds, may be employed in the calculation of the integrated diagnostic value, and where the integrated diagnostic value is determined by calculating the weighted sum of the number of measurements within each range (the resulting sum may be normalized to a suitable value).

Having obtained the integrated risk measure made up of both the measured diagnostic data and the patient risk factor data, a visual indication of the value of the integrated risk measure may be shown on the odontogram for the measured tooth. The visual indication may be provided in a multitude of forms, as described above. Example visual indications include textual markings adjacent to the tooth, the fill or outline colour of a tooth, the fill pattern of a tooth, the intensity of the displayed tooth outline, and the intensity of the fill of a tooth.

As described above, the integrated risk measure may be binned into various categorizations. In a non-limiting example, the categories may include “healthy”, “low risk of developing carious lesions”, “high risk of developing carious lesions”, and “carious lesions detected”. Alternatively, integrated risk measure results may be shown separately, with the odontogram only showing the diagnostic results, as in the aforementioned embodiment. In one example, the user may control the display of data on the odontogram, such that one or both of the diagnostic data and the integrated risk measure data are displayed. The integrated risk measure may be obtained by assessing the diagnostic measurements for a collection of the patient's teeth, and determining an overall diagnostic risk measure that is combined with the patient data risk measure to obtain an overall integrated risk factor, as disclosed in U.S. patent application Ser. No. 12/718,746.

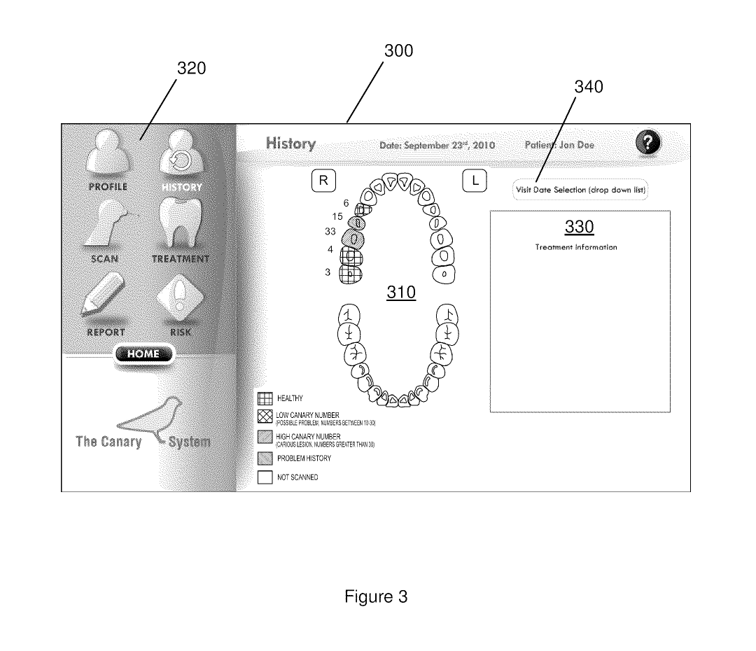

FIG. 3 shows a non-limiting example of a user interface for displaying the odontogram and the oral health status of various scanned teeth. The user interface window 300 includes the odontogram 310, a series of menu labels 320, and an additional window region 330 showing a treatment note relating to the patient. In the embodiment shown in the Figure, the “History” menu label has been selected for reviewing historical results from past patient visits. The “History” screen shown in the figure provides a drop down menu 340 for selecting a specific date or past appointment.

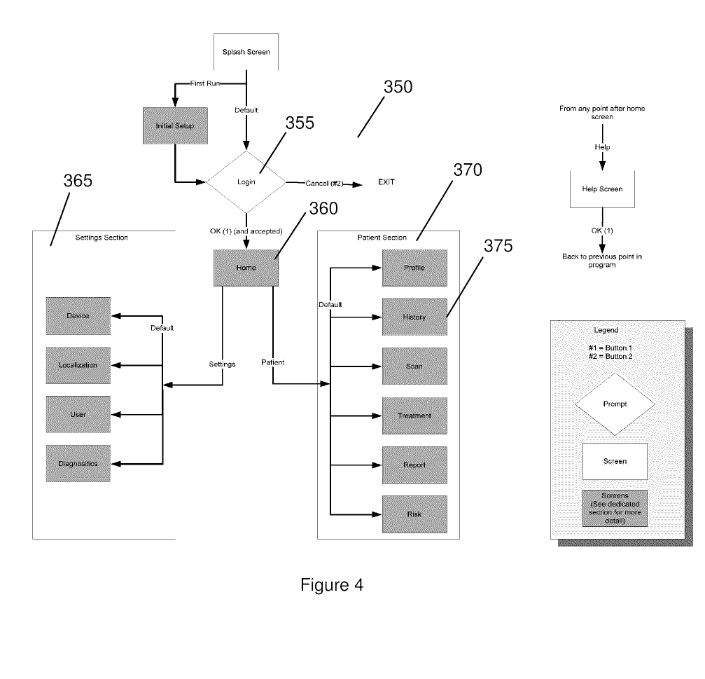

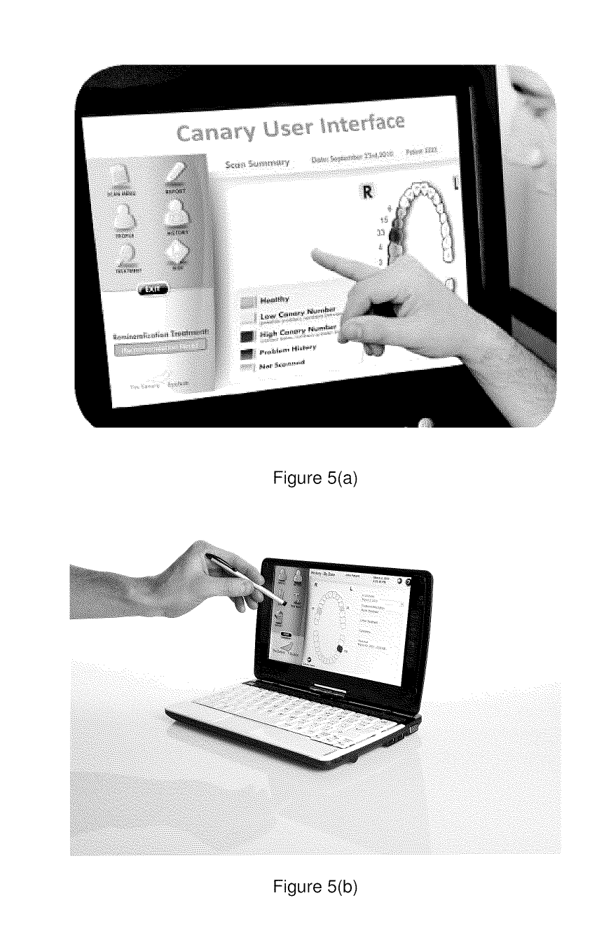

FIG. 4 shows a hierarchical diagram 350 of an exemplary yet non-limiting embodiment of the user interface where the user may navigate among a variety of screens. The user interface comprises a series of welcome and login screens 355, which direct the user to a home screen 360, where the user may select between a series of setting screens 365 and patient/clinical screens 370. For example, the “History” screen shown in FIG. 3 is accessed by selecting the “History” screen 375 from the patient/clinical screens 365. Additional patient screens include the patient profile for entering an managing patient information, a scanning screen for scanning individual teeth using the diagnostic device, a treatment screen for entering treatment notes, a reporting screen for generating a patient encounter report, and a risk screen for entering patient risk factors and/or reviewing a risk assessment generated as described above. The user interface may be provided as a touchscreen interface for rapid and convenient navigation among the various screens, as shown in FIGS. 5(a) and 5(b).

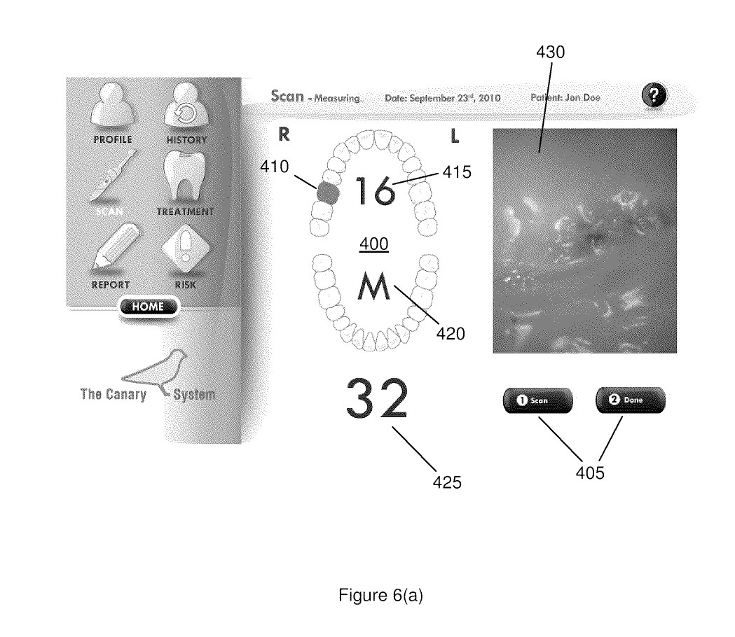

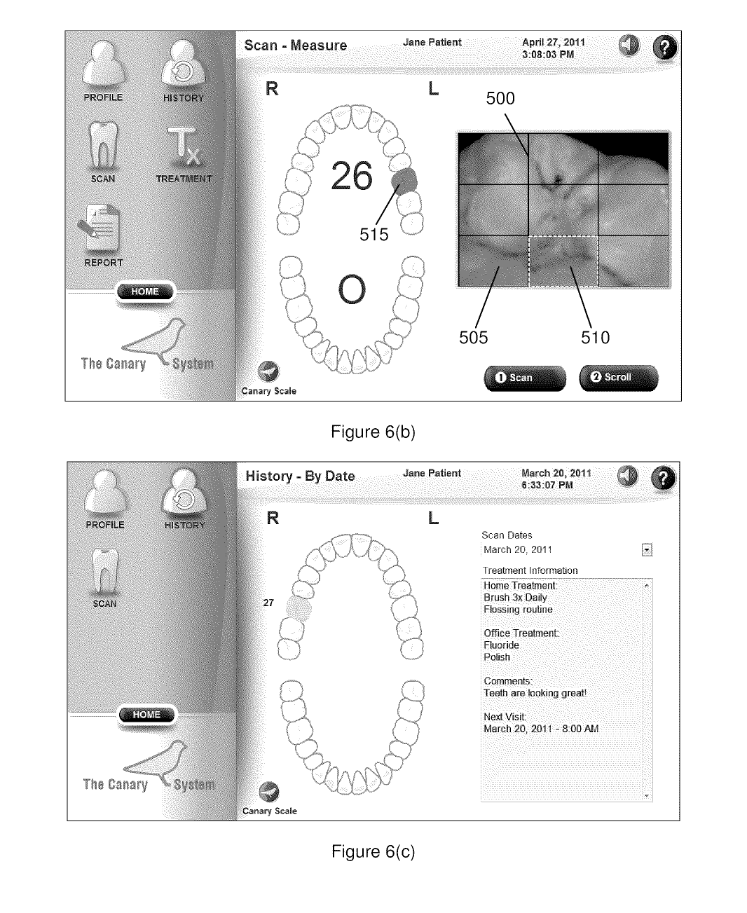

An embodiment of a scanning user interface screen is shown in FIG. 6(a), where the user interface displays an odontogram 400 and scan selection buttons 405. The user interface displays the selected tooth 410 on the odontogram, for example, by colouring or shading the selected tooth as shown in the Figure. Additionally, further information identifying the scanned tooth may be provided, for example by displaying the tooth number 415 using a standard system such as the FDI system. In one embodiment, the display may also identify the scanned surface by an appropriate identifier, such as letter M shown at 420, identifying the scanned surface as a mesial surface. In another embodiment, the user interface may allow the operator to select either primary or a permanent tooth, for example, by touching or activating a touch screen on the diagnostic device console or a separate display.

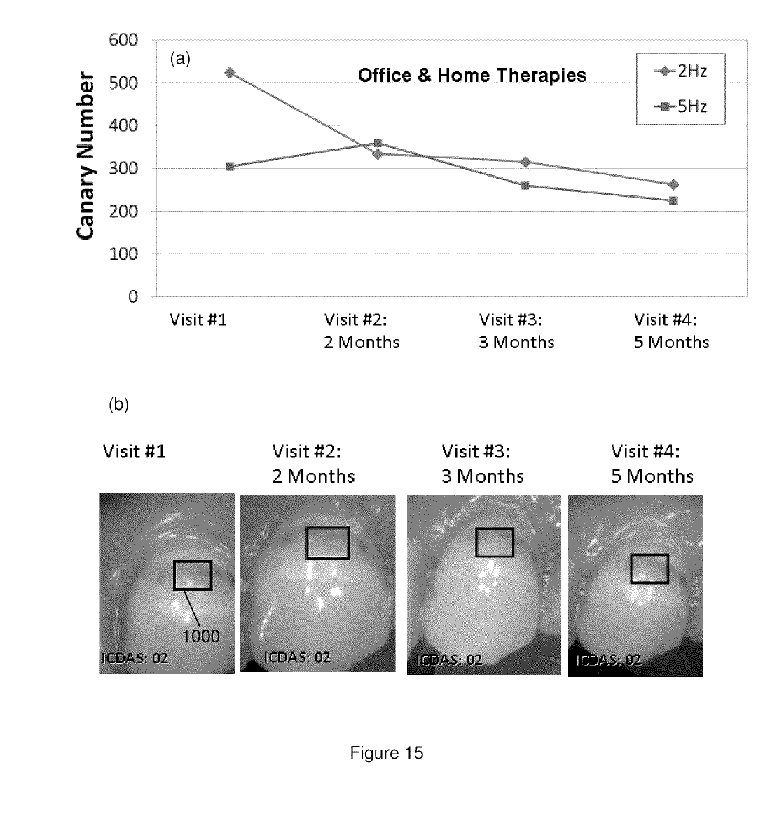

After having obtained the scan results, the results of the scan may be shown in the user interface. In the embodiment shown in the Figure, the Canary number is shown at 425, which displays the highest measured number obtained over the series of measurements included in the scan. Alternatively, the diagnostic values obtained while scanning may be plotted graphically, showing the spatial profile of the measured values (see FIG. 15).

In one embodiment, the user interface also displays an image of the scanned tooth as shown at 430 in FIG. 6(a), so that an image of the scanned tooth may be displayed adjacent to the odontogram. This allows the provider to clearly identify the region of the tooth that was scanned to obtain the result, and facilitates dialogue and treatment discussion between the provider and the patient. The image may be obtained, for example, from the diagnostic device, provided that it is equipped with a camera. In cases where more than one surface of a tooth is measured, one or more images per tooth surface measured may be recorded and optionally displayed.

In general, scanning a tooth surface involves examining an area of the tooth surface to determine whether or not any pathology is present. An objective of scanning is to identify those areas of the tooth surface that require treatment either remineralization or operative intervention (placement of a filling). Considering the specific example of the Canary PTR-LUM system, the beam diameter is approximately 150 microns, but information is gathered from the surrounding tooth structure. The operator may collect data by slowly moving the diagnostic device handpiece around the tooth surface gathering information. Clinical trials have identified an area that needs study usually by its visual characteristics such as:
Brown or white spot on a smooth tooth surface
Stained grooves on the occlusal or biting surface of the tooth
Shadows in the interproximal or contact areas of the tooth
Shadows on the occlusal, buccal or lingual surface beneath the pits and fissure
Contact areas between teeth in general.

In one embodiment, scanning involves first capturing an image of the area to be examined. The operator places the handpiece on the tooth surface and begins the scan. For scanning involving the Canary PTR-LUM system, each scan requires approximately five seconds and is followed by a musical tone and voice prompt with the Canary Number or a request to repeat the scan. Provided that the scan need not be repeated, the probe may be moved to an adjacent site and the scan will begin again automatically. After the operator has finished examining the tooth, the operator may provide input to the user interface that the scan is complete (e.g. selecting, via a touchscreen display, the “done” button of bottoms 405 in FIG. 6(a)) and the scan will end. The operator may conclude the scan with the handpiece measuring the area of tooth that is of concern or has the large Canary number. The operator may then move on to the next surface or next tooth.

In one embodiment, the surface of the tooth may be divided into two or more image elements for scanning. For example, image elements may include the mesial occlusal and disto-occlusal areas. This may be particularly useful for scanning molar teeth. To further facilitate such scanning over multiple areas, a grid system can be overlaid on the tooth image allowing the operator to scan multiple sections of the tooth surface. This allows for multiple measurements to be taken and stored of one tooth surface under examination.

FIG. 6(b) provides an example implementation where a grid 500 is overlaid on the image 505 of the tooth. Grid 500 identifies multiple surface elements that may be optionally scanned, such that a scan within a particular grid element is correlated with the spatial location of the grid element. For example, a user may select a particular grid element to scan, and then scan an area within the grid element as shown by grid 500. This example implementation is shown in FIG. 6(b), where a particular grid element 510 has been selected for scanning. The operator scans the surface of the tooth corresponding to the area shown in element 510 of image 500, and the scan results for the grid element are recorded by the system.

For example, for grid element 510 in FIG. 6(b), a Canary number of 26 is measured and stored in association with grid element 510 (this number may be the maximum Canary number obtained while scanning within the region corresponding to element 510). A provider may subsequently select the tooth 515 in the odontogram and display the Canary readings obtained for any of the grid elements for which measurements were obtained. In one example, diagnostic readings for two or more grid elements may be combined when determining an oral health status of a selected tooth. For example, the user interface may display the highest diagnostic reading (e.g. Canary number in the present case) obtained for all elements, which corresponds to the surface region with the greater pathological problem or risk. This reading may be compared with reference data to determine the oral health status of the selected tooth.

In the above example, up to 9 distinct areas of the tooth surface can be examined and diagnostic measurements can be saved for each grid element. However, it is to be understood that the grid shown in FIG. 6(b) is merely an example of a grid arrangement, and the grid may contain more or less elements than as shown.

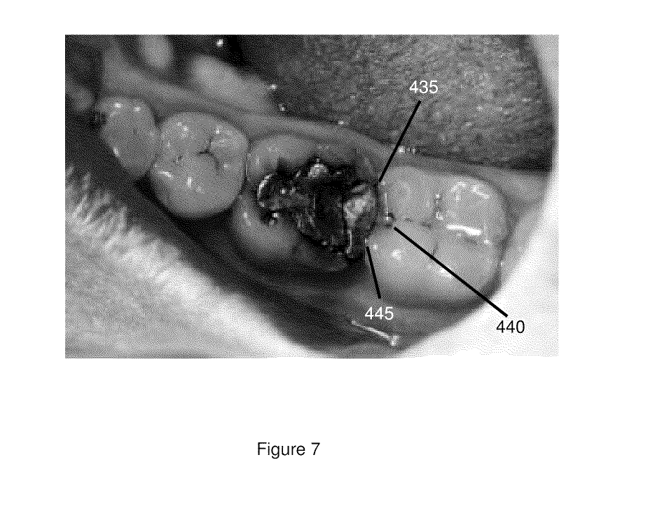

In one example involving the scanning of interproximal areas, scanning typically involves capturing data from adjacent teeth at their respective contact points. As shown in FIG. 7, an operator examines the teeth from three sides, occlusal or biting surface, buccal or outside surface and lingual or tongue or roof of the mouth surface. Scanning involves moving the device around the contact area or ridge on the biting surface and also along the sides of the teeth. These scans may be classified as interproximal scans or lesions in the contact area or contact point. Detection of a lesion at the contact point may involve scanning three surfaces, as shown at 435, 440, and 445 in FIG. 7, and associating these surfaces with a tooth. Accordingly, an interproximal scan may involve occlusal, buccal and lingual scanning at the contact point. In one embodiment, an interproximal scan may be performed by measuring within one or more elements of a grid, as described above.

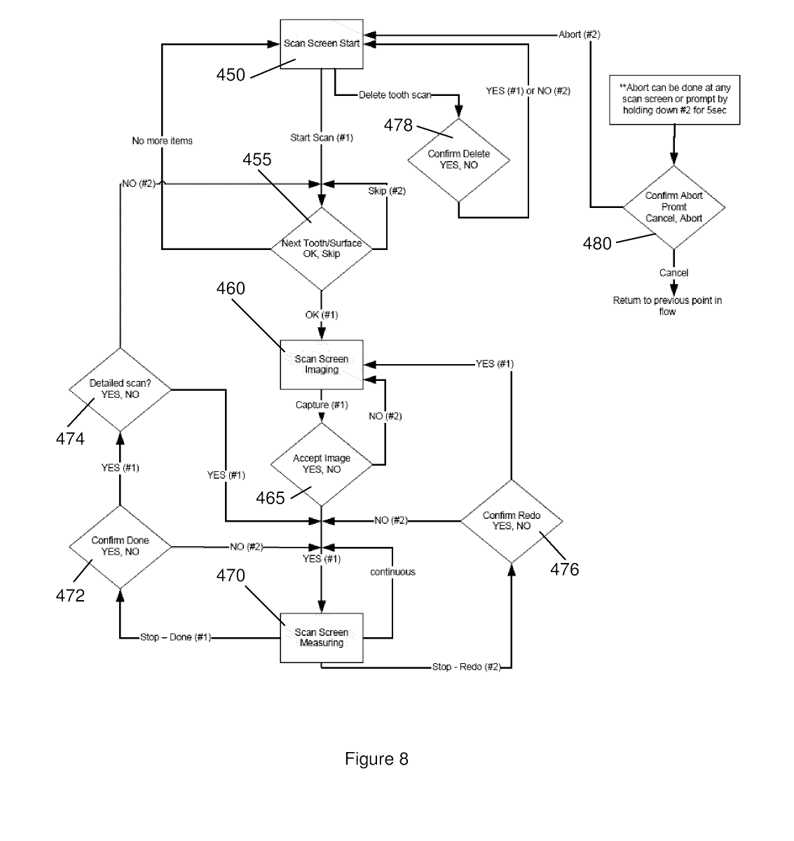

FIG. 8 provides a flow chart illustrating the process of acquiring an image of a selected tooth and performing a diagnostic measurement of scan on the selected tooth. The user is presented with a scanning screen in step 450, and a specific tooth may be selected or skipped in step 455. An image of the selected tooth is then obtained in step 460, followed by a diagnostic scan that is recorded in step 470 provided that the image is accepted in step 465. The flow chart further provides additional steps for confirming or redoing various steps in the process, as shown in FIG. 8 at 472, 474, 476, 478 and 480.

In yet another embodiment, the user interface may include drawing tools, such as lines, arrows, and freeform tools, for annotating an image on a tooth. These tools may be used to illustrate, on the image, the location where the scan is performed. Additional features, such as areas of visible oral health issues such as decay, recession, dental restorations or past dental procedures, may also be annotated.

In one example shown in FIG. 6(c), the user may view a report showing both the results of the scanning as well as the recommended treatments for a particular examination day. This type of report provides the operator/user of the device with information on past examinations and therapeutic interventions.

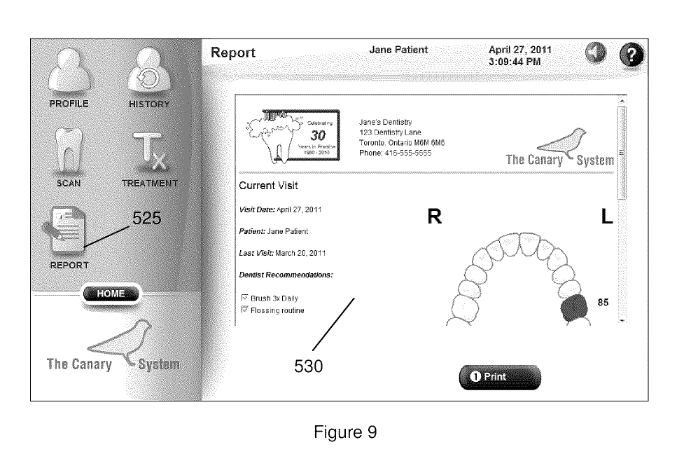

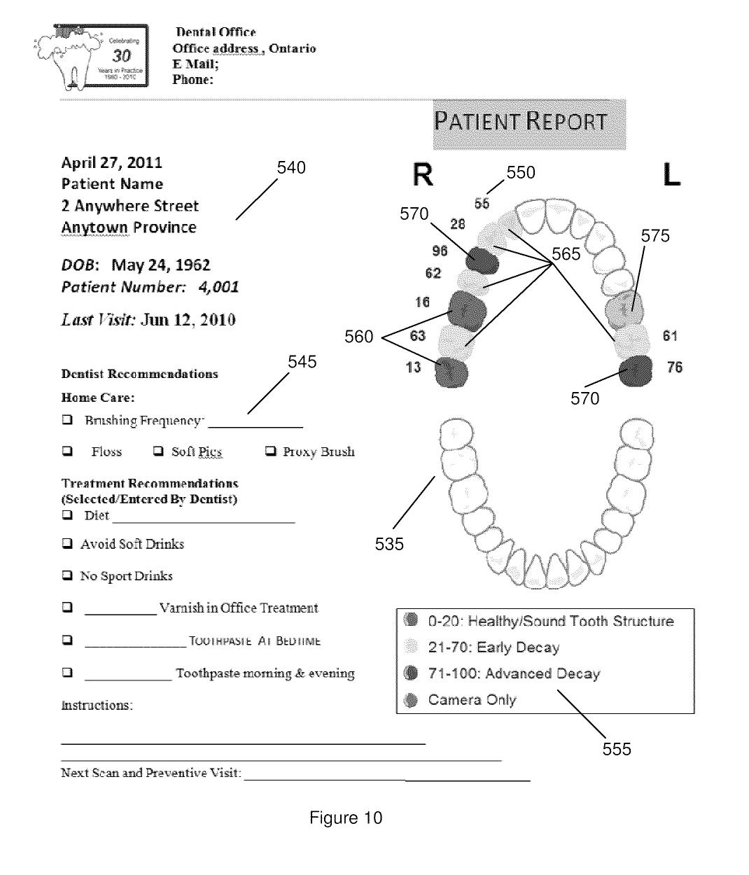

In one example implementation, the user may instruct the system to prepare a report, as illustrated in the example screen shot provided in FIG. 9. Show in FIG. 9 is the selection of a report icon, which generates example report 530 that is shown in full in FIG. 10.

Referring to FIG. 10, the example report includes odontogram 535, patient information 540, and treatment recommendation information 545. Odontogram 535 may be displayed with diagnostic data that is provided on a per tooth basis, as shown in the example where per-tooth Canary numbers 550 are provided for each scanned tooth. It is noted that the present report provides Canary Numbers that are normalized to a scale ranging from zero to 100.

As indicated by legend 555, the results from the diagnostic measurements may be additionally (or alternatively) displayed on a qualitative or semi-quantitative basis. In the example shown in FIG. 10, legend 555 shows that teeth having a low Canary number between 0 and 20 are shown as “Healthy/Sound Tooth Structure” and displayed in odontogram 535 according to a first fill type shown at 560. Teeth having a moderate Canary number between 21 and 70 are shown as “Early Decay” and displayed in odontogram 535 according to a second fill type shown at 565. Teeth having a high Canary number between 71 and 100 are shown as “Advanced Decay” and displayed in odontogram 535 according to a third fill type shown at 570. Additional fill types may be provided to show other types of measurements, such as at tooth 575 for which the only data obtained is in the form of a camera image.

The fill types of the displayed teeth may be provided according to a wide variety of colours, textures, shades, and other features that are visually distinguishable. In one example, the fill types may be different colours that are associated with hazards or risk. For example, the first fill type, which is associated with healthy teeth, may be shown as a shade of green, the second fill type, which is associated with early decay, may be shown as a shade of yellow or orange, and the third fill type, which is associated with advanced decay, may be shown as a shade of red. In another example, the intensity of the colour is correlated with the diagnostic measurement. For example, a very low Canary number of 2 may be shown in bright green, while a Canary number of 18 may be shown in a lighter green colour. It will be understood that there are many different ways of visually conveying risk, inferred oral health problems, or known oral health problems.

FIG. 10 provides one example implementation in which the visual display of the odontogram includes per-tooth oral health status information and diagnostic data. Additionally or alternatively, risk factor data may be displayed on a per tooth basis, either quantitatively, qualitatively, or both, as described above. For example, the report may include per-tooth, or per-tooth surface, integrated risk factor measures that are based on risk factor data and measurements made with a diagnostic device, as described above.

In another example implementation, the user interface may include one or more review screens for reviewing oral health data. The review screens may be useful as a tool for a patient to discuss, for example, a patient's diagnostic results, risk factors, oral health history, treatment history, and planned or suggested treatments and/or interventions. This review process may be effective in improving patient awareness of his or her oral health, and for engaging the patient to take a more active role in maintaining or improving his or her oral health.

According to one example, the review screen may include a graph of time-dependent (e.g. historical) diagnostic and/or risk measure data, which supports tracking of oral health status and the effectiveness of treatments. The review screen may take the form of a screen that is similar to the screen displayed in FIG. 2, 3, 6(a), or 6(b), where selecting a given tooth or tooth surface (such as via a touchscreen interface) causes the system to display historical data for the given tooth or tooth surface. In one example, the historical data may be provided in a tabulated form, where in another example, the historical data may be displayed as a function of time. In another example, selecting a single tooth generates a time dependent plot showing data from all individually scanned tooth surfaces, such as those pertaining to different areas of a grid. Images of the tooth or tooth surface obtained over multiple time points may also be provided. Such an example is described in detail in Example 3 below, where FIG. 15 illustrates the ability of the system to track and store data on the oral health status of a section of tooth over time.

In one embodiment, the time-dependent diagnostic and/or risk measure data may be displayed with information indicating the timing and optionally the nature of therapeutic treatments or interventions. By combining the display of the diagnostic data and/or risk measure data with treatment information, the operator, provider and/or patient may readily assess and/or observe the relationship between treatments and oral health.

In another example implementation, the review screen may be provided in the form of an interactive review screen that displays integrated risk measure information for one or more teeth or tooth surfaces, and also accepts input allowing the operator to vary one or more risk factors. The integrated risk measure information then varies according to the change in the risk factors. Accordingly, a tool may be provided for presenting and communicating a sensitivity analysis, where the sensitivity of a patient's specific per-tooth or per-tooth surface integrated risk measures to changes in one or more risk factors is shown. This may be useful in motivating a patient to make one or more changes relating to the risk factors, such as a change in diet or oral hygiene habits, in order to achieve an improved clinical outcome in a subsequent visit.

The user interface may also include one or more screens whereupon an operator may provide input to record treatment recommendations for a pathological condition identified during a particular patient visit. Using the aforementioned review and tracking screens. As a result, the user interface facilitates the interpretation of the effectiveness of the treatment recommendations to influence or change the numerical value associated with a particular tooth, tooth surface or section of tooth surface, which may be documented in the form of a report showing the outcomes of the treatment recommendations on diagnostic measurements.

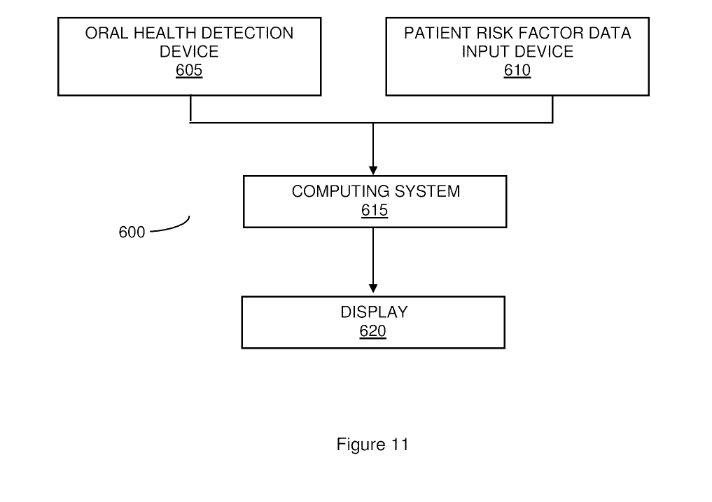

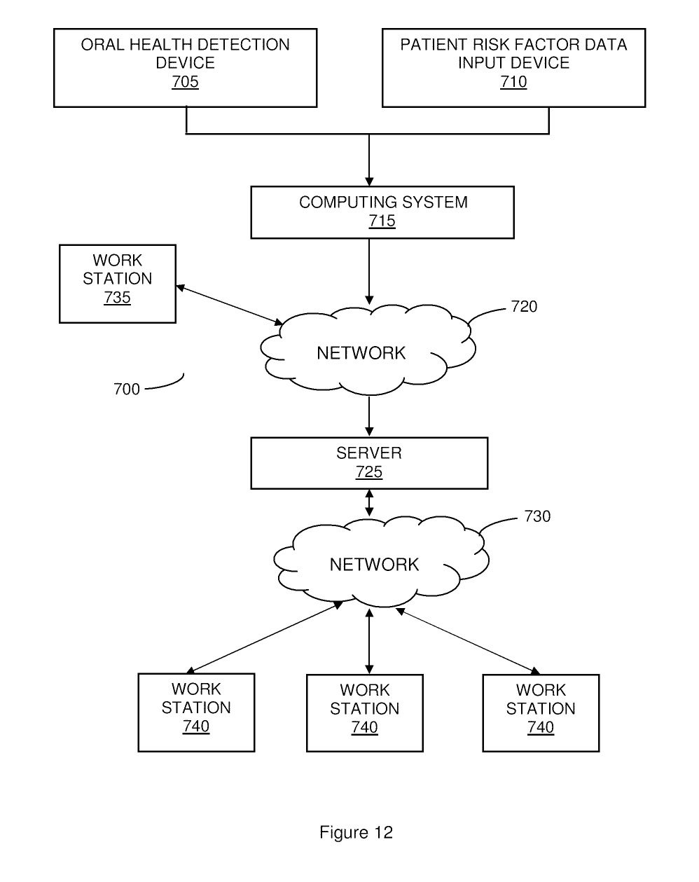

FIGS. 11 and 12 provide non-limiting embodiments showing various computing systems for carrying out the aforementioned embodiments. FIG. 11 provides a schematic of a local system 600 in which the diagnostic device 605 (and optionally input device 610) is connected to a local computing system 615 such as a personal computer, workstation, or local server through a data interface. The interface may be a wired connection, such as a USB connection or a wireless connection, such as through a local WiFi™ network.

Computing system 615 may also be connected to an input device 610 for providing patient risk factor data, as discussed above. In a non-limiting example, the input device may be a keyboard connected to a computer housing processor 615, or may alternatively be an external input device such as a second computer, patient kiosk, or workstation connected to processor 615.

A display 620 is connected to computing system 615 for displaying the oral health status of teeth scanned with the diagnostic device 605, as described in the embodiments listed above. The display may include a monitor directly connected to computing system 615, or may alternatively be provided as an external display device, such as a laptop, netbook, electronic document reader, tablet, smart phone, or other portable media device or built directly into the computing system. Display 620 may enable a user to view the oral health status of selected teeth, and optionally, to control or otherwise interface with the diagnostic device, through a user interface as described above. In one embodiment, the system further comprises a data input device such as a touchscreen or a keyboard. The display may be a touchscreen display. Any or all of system components 610, 615 and 620 may be integrated with diagnostic device 605. For example, diagnostic device 605 may include a display.

In one embodiment, system 600 includes a general purpose computer or any other hardware equivalents. Thus, the system may include at least one processor (CPU/microprocessor), a memory, which may include random access memory (RAM), one or more storage devices (e.g., a tape drive, a floppy drive, a hard disk drive or a compact disk drive), and/or read only memory (ROM), and various input/output devices (e.g., a receiver, a transmitter, a speaker, a display, an imaging sensor, such as those used in a digital still camera or digital video camera, a clock, an output port, a user input device, such as a keyboard, a keypad, a mouse, a position tracked stylus, a position tracked probe, a foot switch, 6-degree input device based on the position tracking of a handheld device, and the like, and/or a microphone for capturing speech commands, etc.).

While some embodiments have been described in the context of fully functioning computers and computer systems, those skilled in the art will appreciate that various embodiments are capable of being distributed as a program product in a variety of forms and are capable of being applied regardless of the particular type of machine or computer readable media used to actually effect the distribution.

Examples of computer-readable media include but are not limited to recordable and non-recordable type media such as volatile and non-volatile memory devices, read only memory (ROM), random access memory (RAM), flash memory devices, floppy and other removable disks, magnetic disk storage media, optical storage media (e.g., Compact Disk Read-Only Memory (CD ROMS), Digital Versatile Disks, (DVDs), etc.), among others. The instructions can be embodied in digital and analog communication links for electrical, optical, acoustical or other forms of propagated signals, such as carrier waves, infrared signals, digital signals, etc.

A machine readable medium can be used to store software and data which when executed by a data processing system causes the system to perform various methods. The executable software and data can be stored in various places including for example ROM, volatile RAM, non-volatile memory and/or cache. Portions of this software and/or data can be stored in any one of these storage devices. In general, a machine readable medium includes any mechanism that provides (i.e., stores and/or transmits) information in a form accessible by a machine (e.g., a computer, network device, personal digital assistant, manufacturing tool, any device with a set of one or more processors, etc.).

Some aspects of the present disclosure can be embodied, at least in part, in software. That is, the techniques can be carried out in a computer system or other data processing system in response to its processor, such as a microprocessor, executing sequences of instructions contained in a memory, such as ROM, volatile RAM, non-volatile memory, cache, magnetic and optical disks, or a remote storage device. Further, the instructions can be downloaded into a computing device over a data network in a form of compiled and linked version.

Alternatively, the logic to perform the processes as discussed above could be implemented in additional computer and/or machine readable media, such as discrete hardware components as large-scale integrated circuits (LSI's), application-specific integrated circuits (ASIC's), or firmware such as electrically erasable programmable read-only memory (EEPROM's).

A networked computing environment 800 is schematically shown in FIG. 12, where oral health diagnostic device 705 (and optionally input device 710) is shown connected to computing system 715. Computing system 715 interfaces with first network 720 and is connected to server 725 for storing, archiving and accessing patient records. A display or workstation 735 may be connected to computing system 715 via first network 720 for displaying the oral health status of teeth scanned with the diagnostic device 705, for example, through a user interface controlled by server 720. Additionally or alternatively, a display with an optional data input means may be directly connected to processor 715, as shown in FIG. 11. Alternatively, computing system 715 may reside beyond first network 720 and may be directly interfaced with or reside within server 725

Server 725 may communicate with one or more workstations 735 through second network 730 for displaying oral health status of scanned teeth on workstations 735, where the display is may be provided via a user interface. In one embodiment, first network 720 comprises a local network, such as a network within a clinical setting, and second network 730 comprises a remote network such as the internet. By providing access to a user interface located on a remote workstation, patients, providers, insurers, and researchers may access relevant oral health status information relating to the scanned teeth of a given patient, for example, provided that suitable credentials are established. In one embodiment, patients may be granted access to their oral health records, and may view the oral health status of their scanned teeth from a remote computing environment. In one embodiment, the user interface accessed by users at workstations 735 and 740 are provided in a web-based or hosted configuration. It is to be understood that workstations 735 and 740 may comprise any computing system adaptable for the display the user interface, including, but not limited to, a laptop, netbook, electronic document reader, tablet, smart phone, or other portable media device.

In one example, the oral health data associated with the system may be stored on a cloud-based server. Such a server, having patient and provider privacy restrictions, would maintain a repository of the data and allow the provider and patient access to the reports once they have provided the appropriate identification and authentication. The oral health provider may therefore have access to not only single patient reports, but reports on all patients in the practice for which diagnostic data has been stored. The reports may provide analysis by one of more measures of interest, such as age, geographic location, teeth with Canary Numbers at certain ranges, outcomes of various preventive and remineralization therapies. In addition the oral health provider may optionally access to billing and utilization reports. The patient may be provided with access to their own personal report containing, for example, historical readings, information on various therapies and the overall outcomes. In addition, access to this data may be granted after having removed patient and/or provider identifiers to support analysis of demographic data, caries disease rate data and outcomes from various therapies on a patient population basis.

The following examples are presented to enable those skilled in the art to understand and to practice embodiments of the present disclosure. They should not be considered as a limitation on the scope of the present embodiments, but merely as being illustrative and representative thereof.

### EXAMPLES

### Example 1

### Utility of PTR-LUM Diagnostic Data

In a PTR or PTR-LUM system, such as The Canary Dental Caries Detection System™, a beam of energy (typically a laser) intensity-modulated at a certain frequency is focused onto the sample surface. The resulting periodic heat flow due to the absorbed optical energy in the material is a diffusive process, producing a periodic temperature rise (distribution) which is called a “thermal wave”. This temperature distribution in turn causes a modulated thermal infrared (black-body or Planck radiation) emission which is used to monitor the material under examination. PTR has the ability to penetrate, and yield information about, an opaque medium well beyond the range of optical imaging. Specifically, the frequency dependence of the penetration depth of thermal waves makes it possible to perform depth profiling of materials.

In PTR applications involving turbid media, such as hard dental tissue, depth information is obtained following optical-to-thermal energy conversion and transport of the incident laser power in two distinct modes: conductively, from a near-surface distance controlled by the thermal diffusivity of enamel (50-500 μm) [Brown W S, Dewey W A, Jacobs H R: Thermal properties of teeth. J Dent Res 1970; 49: 752-754] and radiatively, through blackbody emissions from considerably deeper regions commensurate with the optical penetration of the diffusely scattered laser-induced optical field (several mm). For example, deeper subsurface lesions are possible by using a longer wavelength (830-nm) laser source than a 659-nm probe [Jeon, R. J., Han, C., Mandelis, A., Sanchez, V., Abrams, S. H., “Non-intrusive, Non-contacting Frequency-Domain Photothermal Radiometry and Luminescence Depth Profilometry of Carious and Artificial Sub-surface Lesions in Human Teeth,” Journal of Biomedical Optics 2004, July-August, 9, #4, 809-819].

PTR measurements of artificially induced caries on extracted human teeth have shown that the PTR amplitude increases gradually with increasing demineralization time and decreases after remineralisation. The PTR phase also shows gradual and consistent changes with demineralization and demineralization treatment. This behaviour has been attributed to the higher scatter of the diffuse photon field and to thermal-wave confinement in the form of standing waves in the treated region, accompanied by decreased thermophysical properties (thermal diffusivity and thermal conductivity).

Good correlation of PTR-LUM results with the mineral loss or the lesion depth measured with TMR results has indicated that PTR-LUM is capable of monitoring artificially created carious lesions, their evolution during demineralization, and the reversal of the lesions under the growth of a remineralized surface layer [Jeon R. J., Hellen A Matvienko A Mandelis A., Abrams S. H Amaechi B. T., In vitro Detection and Quantification of Enamel and Root Caries Using Infrared Photothermal Radiometry and Modulated Luminescence. Journal of Biomedical Optics 13(3), 048803, 2008]. The PTR-LUM methodology for dental applications has been extensively studied. Literature reports include applications in depth profiling, early lesion evaluation, caries detection in smooth, occlusal, root and interproximal areas, and theoretical modeling.

One of the main advantages of PTR-LUM is the ability to perform depth profiling through scanning of the excitation source modulation frequency. By selecting a fixed modulation frequency, radiometric measurements at different depths in the enamel can be obtained. The first attempt to apply the depth profilometric capability of PTR-LUM toward the inspection of dental defects was reported by Mandelis et al. [Jean, R. J., Mandelis, A., Abrams, S. H., “Depth profilometric case studies in caries diagnostics of human teeth using modulated laser radiometry and luminescence”, Review of Scientific Instruments, 2003, January, Volume 74 #1, pages 380-383]. In these studies a laser of 488 nm was used as the excitation source. This work showed that the photothermal radiometric signals were anti-correlated with the luminescence signals, as a result of the nature of the two physical signal generation processes. While the PTR amplitude increased for carious lesions the LUM amplitude decreased. The LUM signal results were consistent with previous reports [R. Hibst et al.]. In addition, these studies showed that the radiometric amplitude exhibited much superior dynamic (2 orders of magnitude signal resolution) range to luminescence (a factor of 2 only) in distinguishing between intact and cracked sub-surface structures in the enamel. Furthermore, the radiometric signal (amplitude and phase) produced dental images with much better defect localization, delineation, and resolution than those obtained with modulated luminescence.

Further experimental studies [Jeon, R. J., Han, C., Mandelis, A., Sanchez, V Abrams, S. H., “Non-intrusive, Non-contacting Frequency-Domain Photothermal Radiometry and Luminescence Depth Profilometry of Carious and Artificial Sub-surface Lesions in Human Teeth,” Journal of Biomedical Optics 2004, July-August, 9, #4, 809-819] used excitation sources of 659 and 830 nm to assess the feasibility of PTR-LUM to detect deep lesions. PTR frequency scans over the surface of an occlusal fissure into demineralized enamel and dentin showed higher amplitude than those for healthy teeth, as well as a pronounced curvature in both the amplitude and phase signal channels. These can be excellent markers for the diagnosis of subsurface carious lesions. The results showed that PTR-LUM is able to detect artificial subsurface defects with sharp boundaries at depths greater than 5 mm. In addition PTR exhibited superior sensitivity to the presence of sharp boundaries, as well as to changes in natural demineralized regions of the tooth. These results suggested the possibility to detect carious lesions on both occlusal surfaces and the interproximal area of the tooth [Jeon et al.].

In experimental studies, it was found that PTR Amplitude had a very strong correlation with lesion size and shape. LUM phase provided limited information. PTR Phase provided an indication of operator movement if there was a strong shift in the phase number from the norm. If this occurred, the operator was instructed to re-measure the area.

In an embodiment providing a single unified quantitative indication of oral health from a measurement at a given location, the data from each location is stored as four separate signals; PTR amplitude and phase and LUM amplitude and phase. A unified diagnostic measure is obtained according to the following weighting formula:
PTR Amplitude weighted at 45% of the total value
PTR Phase weighted at 15% of the total value
LUM Phase weighted at 10% of the total value
LUM Amplitude weighted at 30% of the total value
   The four readings are compared to the readings one finds from the healthy enamel surface and/or from a standardized piece of hydroxyapatite. The measured signal number is compared to healthy enamel surface as well. Results from the comparison step may be provided on a fixed scale for each reading, for example, on a scale of 1 to 100 (the scales need not be equal for each reading type), indicating a severity of a condition. The four fixed-scale results are then weighted as described above, providing the operator a ranking or range (for example, on a scale from 1-100) indicating the health of the area examined. The utility of multiple readings in diagnostic assessment with a PTR and LUM detection device was illustrated in Jeon [Jeon et al., “Diagnosis of Pit and Fissure Caries Using Frequency-Domain Infrared Photothermal Radiometry and Modulated Laser Luminescence”, Caries. Res. 38, 497-513, 2004].

In another embodiment, the reading from a single frequency is combined in the following manner: (PTR amplitude×PTR Phase)/(LUM Amplitude×LUM Phase) to create one single reading. Error checking is done by combining the standard deviation from each reading into one number as follows: LUM amplitude×LUM Phase×PTR Amplitude×PTR Phase. The ratio of single reading/combined standard deviation is examined and if the ratio increases dramatically this indicates an error in the reading and this is conveyed to the operator. The single reading is then conveyed to the operator along with its difference from the single reading derived from examining health enamel and healthy teeth.

### Example 2

### Photothermal Radiometric and Luminescence System

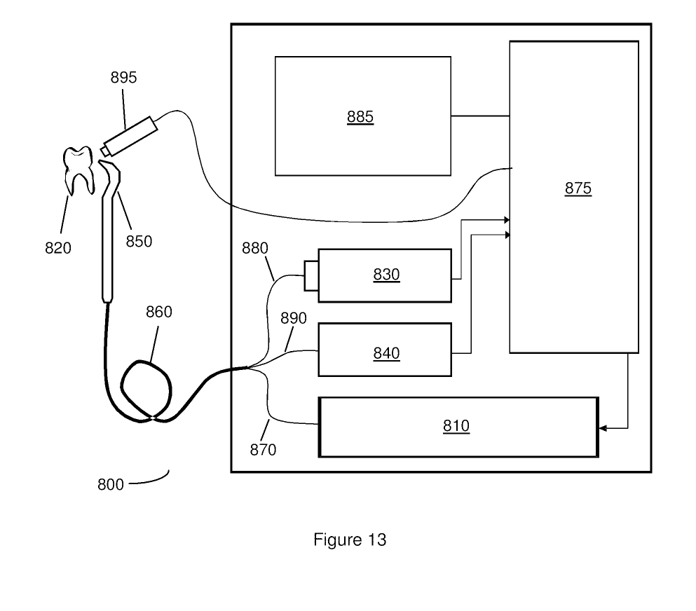

FIG. 13 illustrates non-limiting example of a diagnostic dental device according to one embodiment involving a hybrid PTR-LUM device 300, shown with its main components. Details of the oral health detection device 300 are disclosed in United States Patent Publication No. US20070021670 published on Jan. 25, 2007, which is incorporated herein in its entirety by reference. U.S. Pat. No. 6,584,341 issued to Mandelis et al. entitled “Method and apparatus for detection of defects in teeth”, which is incorporated herein in its entirety by reference, discloses a similar system. In one embodiment, the system includes an optical imaging system, such as, but not limited to, a CCD camera for imaging capture of dental tissue. Other imaging devices may include an infra-red imaging device.

The PTR-LUM system 800 as disclosed in these two US patent publications is used for scanning and data capture of dental tissue. The device is designed for locating and monitoring small early carious lesions, areas of erosion and caries around restorations in a non-invasive fashion. The core technology in device is photothermal radiometry (PTR) and ac luminescence (LUM) as described in other the previously referenced United States patents/applications incorporated by reference. By using PTR and LUM and applying comparison to normal healthy enamel or other mineralized tissue, one can then assess the health of the tooth and monitor ongoing changes. The device can monitor ongoing demineralization (break down of the enamel crystal); early stages of dental caries and remineralization as well as erosion of the tooth surface or caries around dental restorations.

As shown in FIG. 13, the system includes a laser light source 810 for irradiating a portion of a dental surface 820 with an effective wavelength, in which modulated photothermal radiometric signals and modulated luminescence signals are responsively emitted from the dental surface. A first detector 830 detects the emitted modulated luminescence signals, and a second detector 840 detects the emitted modulated photothermal signals. The laser light is emitted from a hand held probe head 850, and a flexible optical fiber bundle 860 having a distal end connected is to the hand held probe head.

The optical fiber bundle includes a first optical fiber 870 having a proximal end in optical communication with the light source and a distal end terminated at the hand held probe head for transmitting light from the light source to a patient's dental tissue by a clinician handling the hand held probe head. The optical fiber bundle additionally includes a plurality of multi-mode optical fibers having distal ends 880 terminated at the hand held probe head and proximal ends optically coupled to the two detectors. A first pre-selected number of the multi-mode optical fibers 880 are near-infrared-transmitting optical fibers for transmitting the modulated luminescence signals to the first detector, and a second pre-selected number of the multi-mode optical fibers 890 are mid-infrared-transmitting optical fibers for transmitting the photothermal radiometry signals to the second detector.

Device 800 includes a demodulator for demodulating the modulated photothermal signals into photothermal phase and amplitude components and the modulated luminescence signals into luminescence phase and amplitude signals. Device 800 further includes a computer processor and or data acquisition and processing card (such as a card offered by National Instruments, Inc., for comparing the photothermal phase and amplitude signals to photothermal phase and amplitude signals of a reference sample and comparing the luminescence phase and amplitude signals to luminescence phase and amplitude signals of a reference sample to obtain differences, if any, between the portion of the dental tissue and the reference sample and correlating any differences with defects in the dental tissue. In FIG. 13 both the demodulator and the computer processor are shown generally as processing unit 875. A computer with touch screen 810, keyboard, and mouse input interface and included and a CCD camera 895 may be included for capturing images of the examined surface of the dental tissue of the patient.

Prior to initiating a scan, information relating to the identification of the patient and the oral tissue (teeth or gum or any other dental tissue and its location) are input through an input device 885 such as a touch screen. The resulting optical signal from sample 820 recorded using the device are collected by the hand piece 850 and optical fiber bundle 860 and sent to detectors 830 and 840. An image of the examined surface is obtained with the CCD camera 895 and sent to the processing unit 875. In an alternative embodiment, an imaging device (such as a CCD camera) may be integrated in the scanning hand piece 850. While a dental technician is operating device 800, the data is captured by scanning the tooth surfaces with an optical probe in hand piece 850.

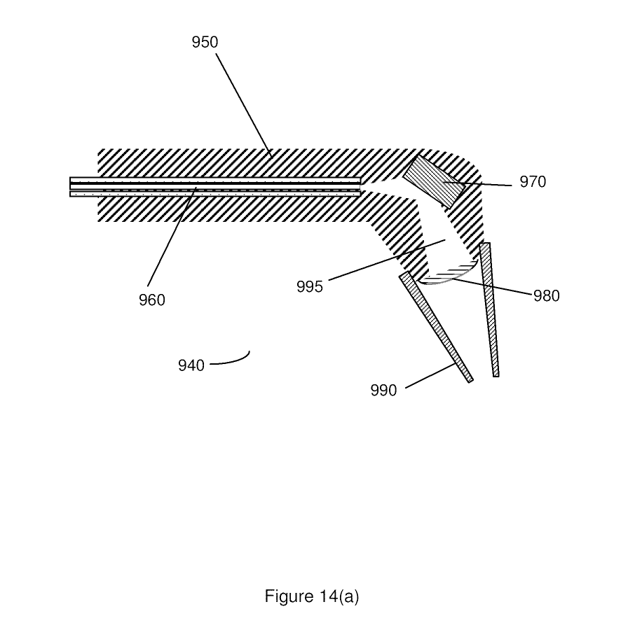

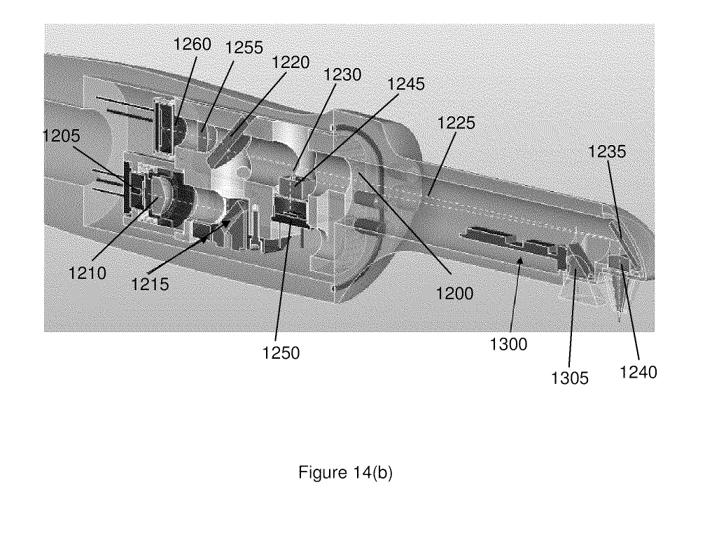

An example schematic of the internal optical configuration for the hand piece 850 is shown in FIG. 14(a). A housing 950 contains the optical fiber bundle 960, mirror 970, lens 980, and spacer 990. The optical fiber bundle 960 delivers the laser light (shown at 995) and collects the scanning results through mirror 970 and lens 980.

FIG. 14(b) illustrates another example of a handpiece for performing measurements based on photothermal radiometry, where the handpiece does not utilize fiber optic beam delivery, as disclosed in co-pending U.S. Provisional Patent Application No. 61/334,436, titled “Handpiece with Integrated Optical System for Photothermal Radiometry and Luminescence Measurements”, and filed on May 13, 2010, which is incorporated herein by reference in its entirety.

A detailed view of the optical apparatus housed within the body portion 1110 and tip portion 1120 is shown in FIG. 14(b). The optical bench 1200 supports semiconductor laser 1205 and lens 1210, which collimates emitted laser light that is subsequently redirected by mirror 1215 towards dichroic window 1220. Semiconductor laser 1205 may be a laser diode having a wavelength of approximately 660 nm for the simultaneous generation of luminescence and photothermal radiation from a tooth surface.

The collimated laser beam is redirected by dichroic window 1220, which may comprises an optical coating having that generate high reflectivity of the incident laser beam while passing thermal radiation. The laser beam propagates in a substantially axial direction into tip portion 1120 along beam path 1225, bypassing pick-off prism 1230 and encountering mirror 1235 at the distal end of tip portion 1120. Mirror 1235 reflects the collimated laser beam towards focusing element 1240, which focuses the laser beam as it emerges from tip portion 1120.

Focusing element 1240 is transparent in the visible light to mid-infrared and has a proper focal length (8.6 mm from the lens surface) for focusing the laser to a spot size of approximately 50 micrometers in average. Focusing element provides the additional role of collecting and substantially collimating both luminescent and photothermal radiation emitted from a tooth surface in response to laser irradiation. While focusing element 1240 is shown as a transmissive optical component, it will be apparent to those skilled in the art that focusing element 1240 and mirror 1235 could be replaced with a single curved off-axis parabolic mirror.

Collected luminescence is directed by mirror 1235 along an axis of tip portion 1120, and a portion of the collected luminescence beam encounters pick-off prism 1230 and is directed towards optical filter 1245 and photodetector 1250. Optical filter 1245 removes unwanted reflected and scattered laser light, and photodetector 1250 is selected to have an optical bandwidth for the detection of the collected luminescence. Photodetector 1250 may be a silicon photodiode, and optical filter 1245 may be an inexpensive color glass filter having a bandwidth and optical density matched to the laser wavelength and power (such as RG 715 Longpass color filter).

As noted above, focusing element 1240 also collects and collimates emitted photothermal radiation, which is reflected by mirror 1235 and directed towards dichroic window 1220. Dichroic window 1220 passes thermal radiation and reflects or absorbs any light which wavelength is below 1.85 micrometers (lower bandwidth of germanium window) including reflected or scattered laser light and luminescence. A suitable material for the absorptive substrate is germanium.

Accordingly, dichroic window with proper coating to enhance transmissivity of mid-infrared and reflectivity of the laser light 1220 passes the collected photothermal radiation, which is subsequently focused by lens 1255 onto infrared detector 1260. Infrared detector may be a sensitive mid-infrared detector, such as a photovoltaic HgCdZnTe detector, with a sensitive spectral region spanning approximately 2 to 5 μm. The infrared detector may be mounted on a thermo-electric cooler for enhanced performance and sensitivity.

As shown in FIG. 14(b), the optical components described above are mounted on optical bench 1200, which may be formed in a lightweight and thermally conductive material such as aluminum. As shown in the FIG. 14(b), the optical bench, while housed within body portion 1110, may be attached to tip portion 1120, thereby enabling rapid and efficient heat sinking into tip portion 1120. Accordingly, tip portion 1120 may also be made from a lightweight and thermally conductive material, such as aluminum. Such an embodiment enables tip portion to act as an efficient air-cooled heat sink for optical components (primarily the laser diode and optionally a TE (thermo-electric) cooler attached to detector 1260) mounted on optical bench 1200. This feature is especially important to optimize the performance of infrared detector 1260, which has a noise floor that is strongly dependent on temperature. Furthermore by attaching the optical bench directly to the rigid tip portion 1120, superior mechanical isolation is obtained relative to attaching the optical components to the body portion 1110.

While the system shown in FIGS. 11 and 12, and their application, have been described and illustrated within the context of an exemplary embodiment, it is to be understood that that numerous embodiments of device system may be made without departing from the scope of the disclosure.

To obtain an output indicative of the oral health of a patient, the oral health detection device, such as the PTR-LUM device discussed above, or a processor or computing system interfaced with the detection device, analyzes the raw data captured from a patient and compares it to a normalized signal for that particular hard tissue. The norm could be either an internally generated function or the signal from a healthy section of hard tissue or a signal generated by hydroxyapatite or other mineralized tissue or commercially produced samples.

Scanning a tooth surface with the device can involve a single frequency, two or more selected frequencies or a frequency scan from 1 Hz to 1000 Hz. In one embodiment, the device is scanned with the option of either 1 or 4 frequencies. The single frequency is used to examine a particular section of tooth surface such as a stained groove. Scanning a tooth with multiple frequencies would create a depth profile picture of the lesion. In one embodiment, scans at 2 Hz, 5 Hz, 20 Hz and 100 Hz provide information on a lesion from near surface to a depth of approximately 4 mm.

### Example 3

### Plotting and Reviewing Historical Data

The present example illustrates the utility of plotting time dependent data associated with a tooth, as described in the aforementioned embodiments. The present example involves a patient having a history of oral health problems, for which remineralization therapy was recommended by a provider.

Prior to initiating remineralization therapy, a first measurement was performed with the Canary™ System, a photothermal diagnostic device as described above. An initial value of the Canary Number for the tooth was determined at two different frequencies (2 Hz and 5 Hz), as shown in FIG. 15(a) (it is noted that the Canary Number shown in FIG. 15(a) are provided on a different scale than the Canary numbers described elsewhere in the present disclosure).

Following the initialization of remineralization treatment, the Canary Number was measured for the tooth during three separate visits at both frequencies. In addition to the diagnostic data, images of the tooth surface were also obtained for each diagnostic measurement, as shown in FIG. 15(b).

Having obtained and recorded historical data associated with the tooth, the provider may review the recorded data and images in order to determine the effectiveness of the therapy. As described above, this may be achieved by selecting the tooth in the odontogram that is displayed in the user interface, which then optionally allows the provider to obtain the plot shown in FIG. 15(a). The corresponding images shown in FIG. 15(b) may also be shown, optionally with an indication of the region scanned, such as rectangle 1000, which may be an image element of a grid (as described above). The plot clearly shows, for the 2 Hz measurements, that the mineralization therapy is effective and improving the oral health associated with the tooth.

In another example, the system may store and display various remineralization therapies employed over time, and display information relating to these therapies in addition to the image and Canary Number data shown in FIG. 15. In addition, as noted above, risk measures may also be tracked and plotted as a function of time for assessing the impact of the prescribed therapy on oral health risk and outcomes.

This improvement in the oral health status of the selected tooth is also reflected in the display of the tooth in the odontogram, as described above. For example, due to the reduction of the Canary number, the displayed tooth in the odontogram may change from red to green in colour over the four visits.

The specific embodiments described above have been shown by way of example, and it should be understood that these embodiments may be susceptible to various modifications and alternative forms. It should be further understood that the claims are not intended to be limited to the particular forms disclosed, but rather to cover all modifications, equivalents, and alternatives falling within the spirit and scope of this disclosure.

1. A computer implemented method of displaying oral health status information of a patient, said method comprising the steps of:
receiving, from an oral health detection device, diagnostic data pertaining to one or more surfaces of a selected tooth;
processing said diagnostic data for each surface of said one or more surfaces to determine an oral health status of said selected tooth; and
displaying an odontogram comprising an indication of said oral health status of said selected tooth.

2. The method according to claim 1 wherein said one or more surfaces comprise at least two surfaces.

3. The method according to claim 1 further comprising the step of displaying, in association with said selected tooth, diagnostic data associated with each surface of said one or more surfaces.

4. The method according to claim 3 wherein said diagnostic data is displayed adjacent to said selected tooth on said odontogram.

5. The method according to claim 3 wherein said step of displaying said diagnostic data associated with each surface of said one or more surfaces comprises displaying, for said each surface, a largest measured value associated with said each surface.

6. The method according to claim 3 wherein said step of displaying said diagnostic data associated with each surface of said one or more surfaces comprises displaying, for said each surface, an average measured value associated with said each surface.

7. The method according to claim 1 further comprising the step of displaying information identifying said each surface.

8. The method according to claim 1 further comprising the step of displaying diagnostic data in association with said selected tooth.

9. The method according to claim 8 wherein said step of displaying diagnostic data in association with said selected tooth comprises displaying a largest measured value associated with said selected tooth.

10. The method according to claim 8 wherein said step of displaying diagnostic data in association with said selected tooth comprises displaying an average measured value associated with said selected tooth.

11. The method according to claim 1 further comprising the steps of receiving one or more images of surfaces of said selected tooth.

12. The method according to claim 11 wherein said diagnostic data is received for each image of said one or more images, such that for a given image, diagnostic data is received in association with a surface shown in said given image.

13. The method according to claim 11 further comprising the steps of:
subdividing an image of said one or more images into two or more image elements; and
displaying said image with said image elements;
wherein said step of receiving said diagnostic data comprises receiving diagnostic data associated with one or more of said image elements.

14. The method according to claim 13 wherein said image elements are arranged in a grid.

15. The method according to claim 13 wherein said one or more surfaces of said selected tooth comprise two or more surfaces shown in different image elements.

16. The method according to claim 11 wherein said image includes a surface selected from the group consisting of occlusal, buccal and lingual surfaces.

17. The method according to claim 1 wherein said step of displaying said indication of said oral health status of said selected tooth comprises modifying a display of said selected tooth on said odontogram.

18. The method according to claim 17 wherein said step of modifying said display of said selected tooth comprises modifying a colour of said tooth, wherein said colour is associated with said oral health status of said selected tooth.

19. The method according to claim 18 wherein said colour is selected to be green for a healthy tooth, and red for an unhealthy tooth.

20. The method according to claim 17 wherein said step of modifying said display of said selected tooth comprises modifying a fill pattern displayed within said tooth.

21. The method according to claim 17 wherein said step of modifying said display of said selected tooth comprises shading said selected tooth with a shading level associated with said oral health status of said selected tooth.

22. The method according to claim 17 further comprising associating said oral health status of said selected tooth with an oral health status category, and modifying said display of said selected tooth according to said oral health status category.

23. The method according to claim 1 wherein said step of processing said diagnostic data to determine said oral health status of said selected tooth comprises combining said diagnostic data with risk factor data to obtain an integrated risk measure, wherein said risk factor data comprises oral health risk factor information associated with said patient.

24. The method according to claim 23 wherein one or more of said risk factor data comprises subjective risk factor data.

25. The method according to claim 23 further comprising the steps of:
receiving input from a user, said input comprising modified risk factor data; and
updating said odontogram to show an effect of said modified risk factor data on said oral health status.

26. The method according to claim 1 further comprising generating a report comprising said odontogram.

27. The method according to claim 1 further comprising
providing, in response to a selection of a given tooth by a user, time-dependent information comprising a historical oral health status associated with said given tooth.

28. The method according to claim 27 further comprising including supplemental treatment information comprising the time at which one or more treatments were administered.

29. The method according to claim 28 wherein said supplemental treatment information further comprises a type said one or more treatments.

30. The method according to claim 27 further comprising plotting said time-dependent information.

31. The method according to claim 27 wherein said time-dependent information comprises diagnostic measurements associated with two or more surfaces of said given tooth.

32. The method according to claim 27 wherein said time-dependent information comprises oral health status information associated with two or more surfaces of said given tooth.

33. The method according to claim 1 wherein said oral health status comprises one of a presence of a dental caries and a formation of a dental caries.

34. The method according to claim 1 wherein said oral health status comprises a presence of white spots or brown spots.

35. The method according to claim 1 wherein said oral health detection device performs a photothermal radiometric measurement or a luminescence detection measurement.

36. The method according to claim 1 wherein said odontogram is displayed on a device selected from the list consisting of a monitor, electronic document reader, tablet, smart phone.

37. The method according to claim 1 wherein said odontogram is displayed on a display associated with said oral health detection device.

38. The method according to claim 1 wherein prior to said step of receiving said diagnostic data, the following steps are performed:
receiving input from a user, said input comprising a request to initiate a measurement with said oral health detection device; and
providing instructions or information to said oral health detection device to initiate said measurement;
wherein said instructions or information are provided through an interface between said oral health detection device and a user interface.

39. The method according to claim 38 further comprising the steps of:
receiving additional input from said user, said additional input identifying a surface of said selected tooth to be measured in said measurement; and
identifying said surface of said selected tooth on said odontogram while said measurement is being performed.

40. The method according to claim 1 wherein said step of displaying said odontogram comprises displaying one or more of primary, permanent and mixed dentition.
